(19) United States
(12) Patent Application Publication
(10) Pub. No.: US 2020/0325434 A1
(43) Pub. Date: Oct. 15, 2020

(54) INCUBATOR PLATE FOR USE IN MICROSCOPY SYSTEM

(71) Applicants: The Trustees of Indiana University, Indianapolis, IN (US); University Houston System, Houston, TX (US)

(72) Inventors: Maria Bondesson Bolin, Bargersville, IN (US); Srijita Mukhopadhyay, Pearland, TX (US); Vrutant Virendra Shah, Pearland, TX (US); Sanat Kumar Upadhyay, Uttar Pradesh (IN)

(21) Appl. No.: 16/843,653

(22) Filed: Apr. 8, 2020

Related U.S. Application Data
(60) Provisional application No. 62/831,281, filed on Apr. 9, 2019.

(51) Int. Cl. C12M 1/00; C12M 1/02

(52) U.S. Cl. CPC C12M 41/14; C12M 41/36; C12M 41/22

(57) ABSTRACT

Disclosed is an incubator plate configured for use in microscopy systems that has at least one housing for a sample vessel and a heating element passageway formed therein.

### PRIORITY CLAIM

This application claims priority under 35 U.S.C. § 119(e) to U.S. Provisional Application Ser. No. 62/831,281, filed 9 Apr. 2019, which is expressly incorporated by reference herein.

### BACKGROUND

Imaging of living cells or small organisms has the potential to provide valuable information on cell proliferation, cell shape changes, cell migratory behaviors, and organismal development. Some living cells and organisms must be maintained at non-ambient temperatures to support these developments. Expensive commercially available temperature control devices are available to enable such non-ambient temperature developments, including microscope stages surrounded by custom-fit Plexiglas boxes, heated plates for culture dishes, and objective warmers for water immersion lenses. These devices strictly control temperature and, in some cases, help control local gas mixtures. Although microscope stage incubators of various designs are commercially available, most are expensive and have other drawbacks.

A need exists in the art for an inexpensive, one-piece, plastic incubator plate that can be manufactured to engage a wide variety of specimen vessels and that maintains a set temperature with minimal fluctuations.

### SUMMARY

A microscopy imaging system according to the present disclosure includes a microscope, a sample vessel, and an incubator module. The microscope is configured for magnification of a specimen. The sample vessel is constructed from transparent material and is configured to hold the specimen. The incubator module is configured to support the sample vessel and to maintain the sample vessel at an elevated temperature during magnification by the microscope.

In illustrated embodiments, the incubator module is specially configured for microscopy and includes a one-piece, unitary plastic incubator plate. The incubator plate is shaped to provide a sample housing onto which the sample vessel is mounted and a heating element passageway formed in the incubator plate. The heating element passageway is configured to hold heated media used to elevate the temperature of the sample vessel during magnification by the microscope.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 shows a first perspective view of an exemplary incubator plate adapted for use in a microscopy imaging system;

FIG. 2 shows a second perspective view of the exemplary incubator plate from FIG. 1;

FIG. 3 shows a partially-diagrammatic view of a first microscopy imaging system incorporating the incubator plate of FIGS. 1 and 2 with a heated water reservoir for heating specimen supported in the incubator plate; and

FIG. 4 shows a partially-diagrammatic view of a second microscopy imaging system incorporating the incubator plate of FIGS. 1 and 2 with an electrically resistive heating element and thermal metallic beads for heating specimen supported in the incubator plate.

### DETAILED DESCRIPTION

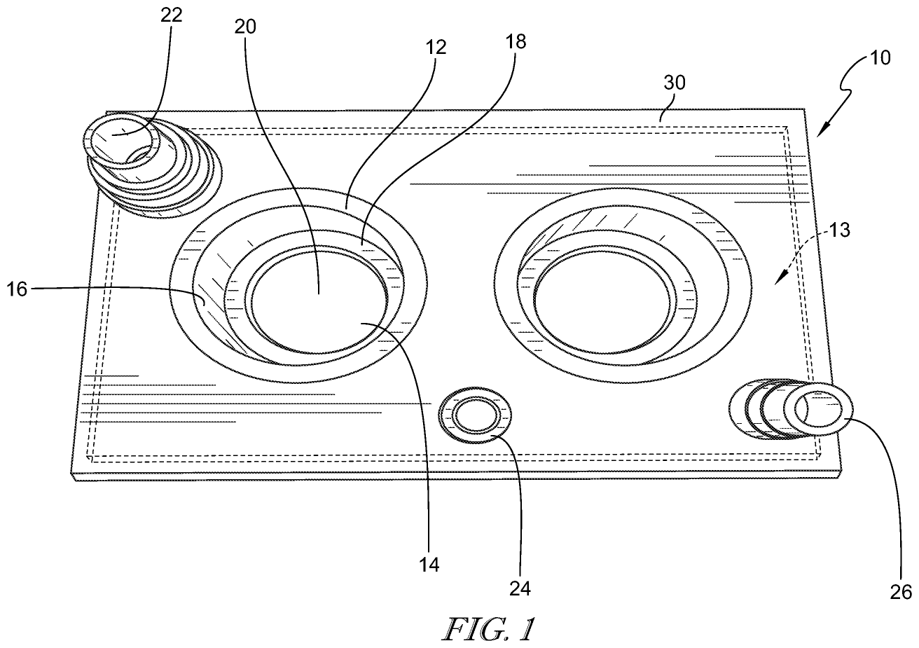

A microscopy imaging system 100 with an incubator plate 10 provides means for imaging cells maintained at non-ambient temperatures as suggested in FIGS. 1-3. In one exemplary embodiment, the system 100 includes a microscope 70 configured for magnification of a specimen, a sample vessel 15 constructed from transparent material configured to hold the specimen, and a microscopy incubator module 11. The incubator module 11 is configured to support the sample vessel 15 and to maintain the sample vessel 15 at an elevated temperature during magnification by the microscope 70.

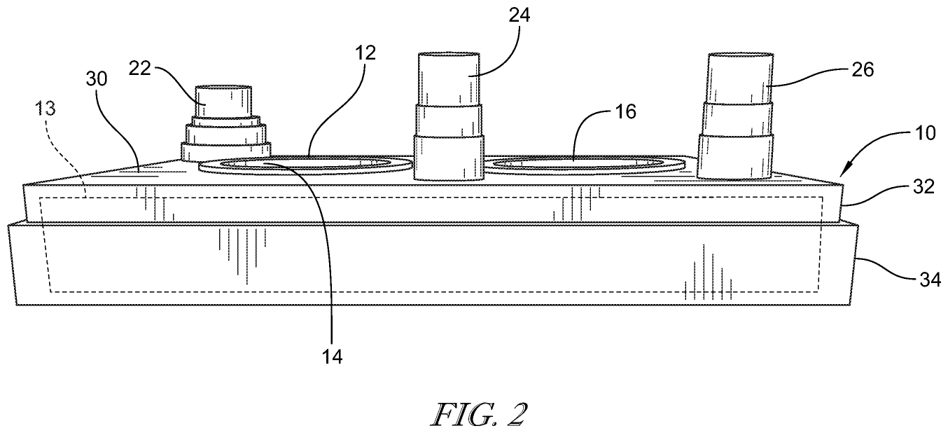

In the exemplary embodiments, the incubator module 11 includes an incubator plate 10 as shown in FIGS. 1 and 2. The incubator plate 10 has a sample housing 12 onto which the sample vessel 15 is mounted and a heating element passageway 13 formed in the incubator plate 10. The heating element passageway 13 is configured to hold heated media used to elevate the temperature of the sample vessel 15 and specimen contained therein during magnification by the microscope 70. It is contemplated that this heating element passageway 13 could also hold cooling medium for chilling the sample vessel 15 as desired by application.

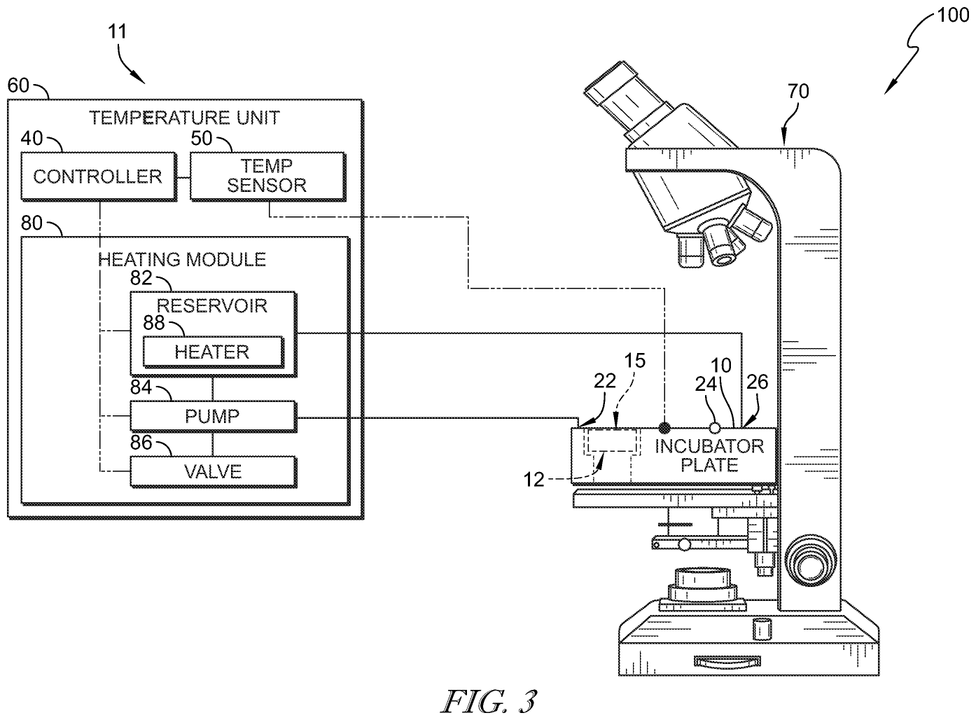

Turning to a first microscopy imaging system 100 shown in FIG. 3, the microscopy incubator module 11 further includes a temperature unit 60 configured to heat a media (water or other media) used in the heating element passageway 13. The temperature unit 60 includes a reservoir 82, a pump 84, a valve 86, and a heater 88. The reservoir 82 is configured to hold the heating media and is fluidly coupled to the heating element passageway 13. The pump 84 is configured to circulate the media from the reservoir 82 through the heating element passageway 13. The valve 86 is optionally used to modulate or open/close fluid connection between the reservoir 82 and the incubator plate 10. The heater 88 is illustratively configured to heat the media in the reservoir 82. It is contemplated that a cooler may be used in place of (or with) the heater 88 in applications where cooling of the specimen vessel 15 is desired.

In the embodiment of FIG. 3 the temperature unit 60 further includes a temperature sensor (or sensors) 50 and a controller 40. The temperature sensor 50 may be a single or multiple sensor package with thermocouples located at various locations. The controller is configured to adjust operation of the pump 84, the valve 86, and/or the heater 88 based on information from the temperature sensor 50. In a simplified arrangement, a basic thermostat without digital controls could also be employed.

In the illustrative embodiment, the incubator plate 10 includes an inlet port 22 and an outlet port 26 interconnected by the heating element passageway 13 as shown in FIGS. 1 and 2. The inlet and outlet ports 22, 26 are provided by tubes that extend upwardly from a planar body of the incubator plate 10.

In the exemplary embodiment, the incubator plate 10 further includes a third port 24 in fluid communication with the heating element passageway 13. The third port 24 is normally closed off but is configured to open in response to a pressure in the heating element passageway 13 exceeding a predetermined threshold. This can be accomplished by providing a thinned section of the incubator plate 10, with a pressure relief valve, or in any other suitable fashion.

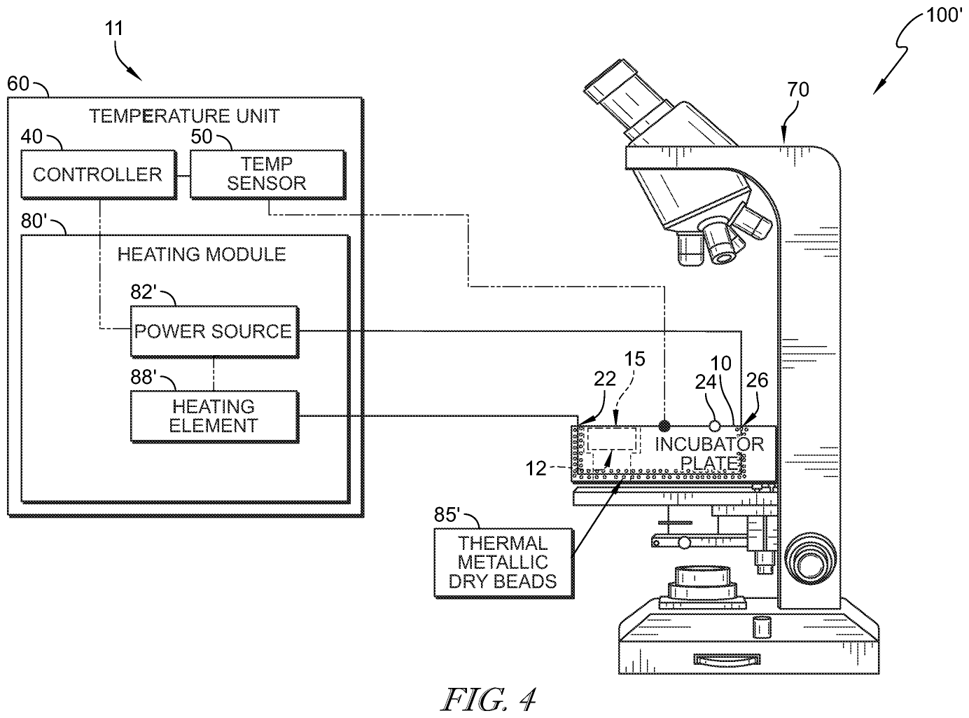

Now looking to FIG. 4 an alternative embodiment of a microscopy imaging system 100′ is shown. In the system 100′, the heating module 80′ differs from module 80 used in the system 100. In particular, heating module 80′ includes an electrical power source 82′ and a heating element 88′. The heating element 88′ may be provided by an electrically resistive wire or the like. In the illustrated design, the module 80′ can also include thermal metallic dry beads 85′ as shown in FIG. 4. The beads 85′ are populated into the passageway 13 of the plate 10 around a resistive wire to distribute heat. It is contemplated that heating element 88′ could be a cooling element or thermal electric device (TED) configured to heat or cool depending on current direction.

FIG. 1 shows an inexpensive, reusable, one-piece, unitary, plastic incubator plate 10 in accordance with the present disclosure. Plastic incubator plate 10 generally includes upper surface 30 and circular sample vessel housing 12. Circular sample vessel housing 12 is configured to receive a 35 mm micro-well glass bottom dish 15. Sample vessel housing 12 includes upper opening 14 sized to receive a sample vessel 15 and lower opening 20. Sample vessel housing 12 has circular side wall 16 and bottom surface 18 that is configured to seat the micro-well glass bottom dish 15. Shape and dimension of the sample vessel housing vary according to the type of sample vessel (Petri dish, glass slide, multiwell plate) to be accommodated. The apparatus can be designed to host one or alternatively more than one of each sample vessel, or also different sample vessels at the same time.

A sample vessel is placed into sample vessel housing 12. Incubator plate 10 is then placed on a light, fluorescence, or confocal microscope stage as suggested in FIGS. 3 and 4. The sample vessel can then be placed into the optical path of the microscope which optical path passes through upper opening 14, through the sample vessel and then through lower opening 20.

Plastic incubator plate 10 further includes serrated inlet port 22, serrated outlet port 26 and serrated third port 24. By circulating heated water through an inner region of plastic incubator plate 10, one or more sample vessels are maintained at a constant target temperature.

Water or other media may be heated externally in a pump 84 or reservoir 82 that has automated temperature control. The externally heated water travels from the pump 84 through tubing and enters an opening in inlet port 22 in plastic incubator plate 10. A regulator valve 86 may be placed between the pump and inlet port 22 as an added water flow control measure. The heated water enters the incubator plate passageway 13 and circulates through inner region of plastic incubator plate 10 before exiting through an opening in outlet port 26 and then into tubing that conveys the waste water to appropriate disposal or recycle means. Third port 24 allows water to exit incubator plate 10 if water pressure exceeds a certain threshold.

In embodiments of the present disclosure, circulating water can be replaced with thermal metallic dry beads 85′ which are inserted into incubator 10 via the ports and then uniformly spread throughout the interior plate space. A heating wire connected to a power source is inserted through inlet port. In addition, a metallic probe connected to a thermostat is inserted through outlet port to maintain a desired temperature. A secondary thin probe is optionally attached to the bottom center of a sample vessel to measure the actual temperature.

FIG. 2 shows another view of the one-piece, unitary, plastic incubator plate 10 in accordance with the present disclosure. Plastic incubator plate 10 generally includes upper surface 30, upper side surface 32, lower side surface 34 and sample vessel housing 12. Sample vessel housing 12 includes central upper opening 14 sized to receive a sample vessel (not shown) and central lower opening (not shown). Sample vessel housing 12 has circular side wall 16 and bottom surface 18 (18 shown in FIG. 1) that is configured to seat the sample vessel. Plastic incubator plate 10 further includes serrated inlet port 22, serrated safety port 24 and serrated outlet port 26.

The plastic incubator plates 10 may be manufactured by a variety of methods known to one of ordinary skill in the art such as by conventional manufacturing or three-dimensional printing. One of ordinary skill in the art recognizes that the specific manufacturing steps including, but not limited to, selective deposition, jetting, fused deposition modeling, multijet modeling and other techniques may be combined in different ways to prepare the inventive plastic incubator plates.

For imaging of living cells or small organisms by microscopy over extended periods of time, the temperature may need to be optimal for the cells or organisms imaged. This can be obtained through the use of an incubator that is mounted on the microscope, or by a heated stage. Both these options are expensive. Designs in accordance with the present disclosure include an incubator plate 10, sometimes called tissue culture well plate, with temperature control through 3D printing. Some embodiments integrate circulating water to control the temperature. Other embodiments incorporate electrical heating.

The present disclosure provides a incubator plate 10, sometimes referred to as a THERMOCONTROL plate, that holds microscope dishes and controls the temperature of them, and thus it replaces the need for an expensive microscope incubator. Exemplary incubator plates 10 that form part of the disclosed system may be manufactured by 3D printing and may have two wells that can fit two commercially available 35 mm micro-well glass bottom dishes, commonly used for confocal microscopy. The dimensions of the exemplary incubator plate 10 is 127 mm×84 mm×18 mm. The plate also has an inlet 22 and two outlets 24, 26. The material used for 3D-printing of prototype plates 10 was Visijet X, a plastic material with a melting temperature above 70° C. All the designs were constructed using the TinkerCad software, regularly used for 3D-printing, and were also double checked for any leakages in the design using the Cura software.

In a first design, temperature can be controlled by circulating water. The circulating water can maintain a uniform heat distribution across the entire plate 10. However, the one consideration of the system 100 with water was the risk of water leakage, in particular when mounted on the confocal microscope stage top. In a second design, water was replaced with thermal metallic dry beads 85′, specifically LAB ARMOR beads, which are manufactured and patented by Sheldon Manufacturing in August 2017, Oregon, USA. These are designed to replace water and they transfer the heat to the contained material. Of course other thermally conductive materials may also be used including various types of polymers.

These thermal metallic beads 85′ may be inserted into the plate via the inlet and the outlet and uniformly spread inside the plate 10. A heating wire 88′ may also be inserted through the inlet 22 and connected to a power supply 82′. A temperature sensor 50 provided by a metallic probe can be inserted in the outlet 26 and connected to a thermostat (controller 40), to maintain a desired temperature of 28° C.-29° C., an optimum temperature required for growth of the zebrafish embryos, or 37° C. for cells. Cooling applications may use thermal electric devices TEDs in place of the heating wire 88 and/or beads 85′ to heat or cool depending on the direction of current applied.

A secondary thin probe was attached to the bottom at the center of the glass plate to record the actual temperature. An offset of +/−1.6° C. was set in the thermostat to obtain the required temperature.

Cost of the incubator plate 10 is very low. It can be reused multiple times for various experiments by switching out the disposable 35 mm dishes that it holds. Secondly, it is a simple, small portable equipment that can be carried easily and moved around from one place to the other with the minimum hassle. Thirdly, there is no inspection and maintenance cost of the product. It is a small, simple plate that fits on the microscope stage. It requires no additional monitoring of the setup. It is user-friendly and is easy to handle. It can be used for imaging of both cells and small organisms.

The outer dimensions of the incubator plate 10 (width and length in particular) are sized to fit most microscope stages. Accordingly, the plate is not only placed on top of the stage, but actually is attached so as to be fixed in place.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

1. A microscopy imaging system, the system comprising
a microscope configured for magnification of a specimen,
a sample vessel constructed from transparent material configured to hold the specimen, and
a microscopy incubator module configured to support the sample vessel and to maintain the sample vessel at an elevated temperature during magnification by the microscope, wherein the incubator module includes an incubator plate having a sample housing onto which the sample vessel is mounted and a heating element passageway formed in the incubator plate that is configured to hold heated media used to elevate the temperature of the sample vessel during magnification by the microscope.

2. The system of claim 1, wherein the incubator plate includes an inlet port and an outlet port interconnected by the heating element passageway.

3. The system of claim 2, wherein the incubator plate is provided by a one-piece monolithic component.

4. The system of claim 3, wherein the incubator plate comprises polymeric materials configured to withstand temperatures greater than about 70 degrees C.

5. The system of claim 2, wherein the microscopy incubator module includes a temperature unit configured to heat a media in the heating element passageway.

6. The system of claim 5, wherein the temperature unit includes a reservoir configured to hold the media and fluidly coupled to the heating element passageway, a heater configured to heat the media, and a pump configured to circulate the media from the reservoir through the heating element passageway.

7. The system of claim 6, wherein the incubator plate includes a third port in fluid communication with the heating element passageway, and the third port is normally closed off but is configured to open in response to a pressure in the heating element passageway exceeding a predetermined threshold.

8. The system of claim 6, wherein the temperature unit includes a temperature sensor and a controller, and the controller is configured to adjust operation of at least one of the heater and the pump based on information from the temperature sensor.

9. The system of claim 5, wherein the temperature unit includes a resistive heating element that extends into the heating element passageway configured to heat up in response to application of electrical current along the resistive wire.

10. The system of claim 9, wherein the heating element passageway is populated with thermal metallic beads adapted to be heated by the resistive heating element.

11. The system of claim 9, wherein the temperature unit includes a temperature sensor and a controller, and the controller is configured to apply current to the resistive heating element based on information from the temperature sensor.

12. The system of claim 1, wherein the sample housing is formed by an upper opening and a lower opening in the incubator plate that cooperate to provide an aperture through the entire incubator plate into which a light source included in the microscope can shine directly onto the sample vessel, and wherein the upper opening is larger than the lower opening such that a bottom surface of the upper opening is created to support the sample vessel when mounted in the incubator plate.

13. The system of claim 12, wherein the upper opening is round with a first diameter, the lower opening is round with a second diameter, and the first diameter is larger than the second diameter.

14. A microscopy incubator module configured to support a sample vessel during viewing by a microscope and to maintain the sample vessel at non-ambient temperature during magnification by the microscope, the microscopy incubator module comprising
an incubator plate formed as a one-piece monolithic component,
wherein the incubator plate is shaped to include a sample housing configured to receive the sample vessel and a heating element passageway that is configured to hold heated media used to elevate the temperature of the sample vessel during magnification by the microscope.

15. The microscopy incubator module of claim 14, wherein the sample housing is formed by an upper opening and a lower opening that cooperate to provide an aperture opening across the entire incubator plate through which the sample vessel may be illuminated by a light source included in the microscope.

16. The microscopy incubator module of claim 15, and wherein the upper opening is larger than the lower opening such that an intermediate surface is created to support the sample vessel when mounted in the incubator plate.

17. The microscopy incubator module of claim 16, wherein the upper opening is round with a first diameter, the lower opening is round with a second diameter, and the first diameter is larger than the second diameter such that a bottom surface of the upper opening provides the intermediate surface configured to support the sample vessel when mounted in the incubator plate.

18. The microscopy incubator module of claim 14, wherein the incubator plate includes an inlet port and an outlet port that each extend outwardly from a planar portion of the incubator plate, and wherein the inlet port and the outlet port are interconnected by the heating element passageway.

19. The microscopy incubator module of claim 18, wherein the incubator plate includes a third port in fluid communication with the heating element passageway, and the third port is normally closed off but is configured to open in response to a pressure in the heating element passageway exceeding a predetermined threshold.

20. The microscopy incubator module of claim 14, wherein the heating element passageway is populated with thermal metallic beads adapted to be heated by an electrically resistive heating element.
